(19) United States
(12) Patent Application Publication
(10) Pub. No.: US 2025/0134661 A1
(43) Pub. Date: May 1, 2025

(54) EXPANDABLE MESH SHEATH WITH PULL WIRES

(71) Applicant: Medtronic, Inc., Minneapolis, MN (US)

(72) Inventors: Jake Dunlea, Galway (IE); Andrea Quero Quero Martin, Galway (IE); David Francis, Claremorris (IE); Mike Francis Conerney, Loughrea (IE)

(21) Appl. No.: 18/911,435

(22) Filed: Oct. 10, 2024

Related U.S. Application Data
(60) Provisional application No. 63/593,628, filed on Oct. 27, 2023.

(51) Int. Cl. A61F 2/24

(52) U.S. Cl. CPC A61F 2/2439; A61F 2/2436

(57) ABSTRACT

An introducer apparatus is provided for delivering a heart valve prosthesis to a treatment site. The introducer apparatus includes a hub including a central passage and an engagement member. The introducer apparatus includes a sheath attached to the hub and including a first cross-sectional dimension. A plurality of wires extend between a first end and an opposing second end. The first end is attached to a distal sheath end of the sheath and the second end is attached to the engagement member. The engagement member moves the plurality of wires and the distal sheath end between an extended position, in which the sheath includes the first cross-sectional dimension, and a retracted position, in which the sheath includes a second cross-sectional dimension that is greater than the first cross-sectional dimension. Methods of delivering a heart valve prosthesis to a treatment site are provided.

### CROSS-REFERENCE TO RELATED APPLICATIONS

This application claims the benefit of U.S. Provisional Patent Application Ser. No. 63/593,628, filed Oct. 27, 2023, the entire content of which is incorporated herein by reference.

### FIELD

The present disclosure relates generally to an expandable mesh sheath and, more particularly, to an expandable mesh sheath with one or more wires.

### BACKGROUND

It is known to provide a prosthetic heart valve assembly for implanting a heart valve prosthesis within a target site of the vasculature of a patient. The heart valve prosthesis can be moved from a radially-contracted position to a radially-expanded position.

### SUMMARY

The following presents a simplified summary of the disclosure to provide a basic understanding of some aspects described in the detailed description.

In aspects, an introducer apparatus is provided for delivering a heart valve prosthesis to a treatment site. The introducer apparatus comprises a hub comprising a central passage and an engagement member. The introducer apparatus comprises a sheath attached to the hub and comprising a first cross-sectional dimension along a plane that is perpendicular to a central axis of the sheath. The introducer apparatus comprises a plurality of wires extending between a first end and an opposing second end. The first end is attached to a distal sheath end of the sheath and the second end is attached to the engagement member. The engagement member is configured to move the plurality of wires and the distal sheath end between an extended position, in which the sheath comprises the first cross-sectional dimension, and a retracted position, in which the sheath comprises a second cross-sectional dimension that is greater than the first cross-sectional dimension.

In aspects, the engagement member is attached to a body portion of the hub, the engagement member configured to move relative to the body portion.

In aspects, the engagement member comprises a first engagement portion that is rotatable relative to the body portion, the first engagement portion circumferentially surrounding the central passage; and a second engagement portion received within the first engagement portion, the second engagement portion configured to move axially relative to the first engagement portion.

In aspects, the second end of the plurality of wires are attached to the second engagement portion such that the plurality of wires extend along the sheath.

In aspects, the engagement member circumferentially surrounds the body portion.

In aspects, the body portion defines at least one wire opening extending radially through the body portion between the central passage and an outer radial side of the body portion, the plurality of wires extending through the at least one wire opening such that the second end is attached to the engagement member.

In aspects, the engagement member is configured to rotate relative to the body portion, and wherein a locking mechanism contacts the engagement member and the body portion to selectively prevent rotation of the engagement member relative to the body portion.

In aspects, the engagement member comprises a first engagement projection and a second engagement projection that are spaced apart to define a first engagement channel between the first engagement projection and the second engagement projection.

In aspects, the body portion comprises a first body projection that is configured to be received within the first engagement channel such that the engagement member and the body portion are configured to move relative to one another between: a first position, in which the engagement member is spaced apart from the body portion such that the first body projection is not received within the first engagement channel, and at least one wire of the plurality of wires extends between the engagement member and the body portion; and a second position, in which the first body projection is received within the first engagement channel, and at least one wire of the plurality of wires extends within the first engagement channel.

In aspects, an introducer apparatus is provided for delivering a heart valve prosthesis to a treatment site. The introducer apparatus comprises a hub comprising a body portion surrounding a central passage, and an engagement member attached to the body portion and configured to move relative to the body portion. The introducer apparatus comprises a sheath attached to the hub. The sheath comprises a lumen in communication with the central passage. The sheath comprises a first cross-sectional dimension along a plane that is perpendicular to a central axis of the sheath. A plurality of wires extend between a first end and an opposing second end. The first end is attached to a distal sheath end of the sheath and the second end is attached to the engagement member. The engagement member is configured to move the plurality of wires and the distal sheath end from an extended position, in which the sheath comprises the first cross-sectional dimension and a first length, to a retracted position, in which the sheath comprises a second cross-sectional dimension, which is greater than the first cross-sectional dimension, and a second length, which is less than the first length.

In aspects, the engagement member comprises: a first engagement portion that is rotatable relative to the body portion, the first engagement portion circumferentially surrounding the central passage; and a second engagement portion received within the first engagement portion, the second engagement portion configured to move axially relative to the first engagement portion, the second end of the plurality of wires attached to the second engagement portion such that the plurality of wires extend along the sheath.

In aspects, the engagement member circumferentially surrounds the body portion and is configured to rotate relative to the body portion, and wherein the body portion defines at least one wire opening extending radially through the body portion between the central passage and an outer radial side of the body portion, the plurality of wires extending through the at least one wire opening such that the second end is attached to the engagement member.

In aspects, the engagement member comprises a first engagement projection and a second engagement projection that are spaced apart to define a first engagement channel between the first engagement projection and the second engagement projection, and wherein the body portion comprises a first body projection that is configured to be received within the first engagement channel, at least one wire of the plurality of wires extending between the engagement member and the body portion.

In aspects, methods of delivering a heart valve prosthesis to a treatment site comprise providing a sheath attached to a hub, the sheath comprising a first cross-sectional dimension along a plane that is perpendicular to a central axis of the sheath. Methods comprise moving a plurality of wires that are attached to a distal sheath end of the sheath in a proximal direction toward the hub. Methods comprise increasing a cross-sectional dimension of the sheath.

In aspects, moving the plurality of wires comprises rotating a first engagement portion relative to a body portion of the hub such that a second engagement portion, which is received within the first engagement portion, moves axially relative to the first engagement portion.

In aspects, the plurality of wires are attached to the second engagement portion such that the axial movement of the second engagement portion causes the plurality of wires and the distal sheath end of the sheath to move in the proximal direction.

In aspects, the plurality of wires extend through at least one wire opening of a body portion of the hub and are attached to an engagement member of the hub.

In aspects, moving the plurality of wires comprises rotating the engagement member relative to the body portion such that the plurality of wires and the distal sheath end of the sheath move axially in the proximal direction.

In aspects, at least one wire of the plurality of wires is positioned between an engagement member of the hub and a body portion of the hub, the at least one wire extending along a linear travel path.

In aspects, moving the plurality of wires comprises moving the engagement member and the body portion together to compress the at least one wire such that the at least one wire extends along a non-linear travel path.

Additional features and advantages of the aspects disclosed herein will be set forth in the detailed description that follows, and in part will be clear to those skilled in the art from that description or recognized by practicing the aspects described herein, including the detailed description which follows, the claims, as well as the appended drawings. It is to be understood that both the foregoing general description and the following detailed description present aspects intended to provide an overview or framework for understanding the nature and character of the aspects disclosed herein. The accompanying drawings are included to provide further understanding and are incorporated into and constitute a part of this specification. The drawings illustrate various aspects of the disclosure, and together with the description explain the principles and operations thereof.

### BRIEF DESCRIPTION OF THE DRAWINGS

These and other features, aspects and advantages are better understood when the following detailed description is read with reference to the accompanying drawings, in which:

FIG. 1 schematically illustrates example aspects of a transcatheter heart valve prosthesis in accordance with aspects of the disclosure;

FIG. 2 illustrates a top-down view of the transcatheter heart valve prosthesis in accordance with aspects of the disclosure;

FIG. 3 illustrates a side view of a delivery assembly for delivering the transcatheter heart valve prosthesis in accordance with aspects of the disclosure;

FIG. 4 illustrates a side view of the delivery assembly for delivering the transcatheter heart valve prosthesis in accordance with aspects of the disclosure;

FIG. 5 illustrates an introducer apparatus in accordance with aspects of the disclosure;

FIG. 6 illustrates an introducer apparatus in accordance with aspects of the disclosure;

FIG. 7 schematically illustrates a side view of the transcatheter heart valve prosthesis positioned at a treatment site in accordance with aspects of the disclosure;

FIG. 8 illustrates a side view of an introducer apparatus comprising a sheath in an extended position, in accordance with aspects of the disclosure;

FIG. 9 illustrates a side view of the introducer apparatus comprising the sheath in a retracted position;

FIG. 10 illustrates a side view of a first embodiment of the introducer apparatus, in accordance with aspects of the disclosure;

FIG. 11 illustrates a sectional view similar to FIG. 11 of the introducer apparatus, in accordance with aspects of the disclosure;

FIG. 12 illustrates a sectional view of the introducer apparatus with the sheath in the extended position, in accordance with aspects of the disclosure;

FIG. 13 illustrates a sectional view of the introducer apparatus with the sheath in the retracted position, in accordance with aspects of the disclosure;

FIG. 14 illustrates a side view of a second embodiment of the introducer apparatus, in accordance with aspects of the disclosure;

FIG. 15 illustrates a sectional view similar to FIG. 14 of the introducer apparatus, in accordance with aspects of the disclosure;

FIG. 16 illustrates a sectional view similar to FIG. 15 of the introducer apparatus, in accordance with aspects of the disclosure;

FIG. 17 illustrates a sectional view of the introducer apparatus with the sheath in the extended position, in accordance with aspects of the disclosure;

FIG. 18 illustrates a sectional view of the introducer apparatus with the sheath in the retracted position, in accordance with aspects of the disclosure;

FIG. 19 illustrates a side view of a third embodiment of the introducer apparatus, in accordance with aspects of the disclosure;

FIG. 20 illustrates a side view of the introducer apparatus similar to FIG. 19, in accordance with aspects of the disclosure; and

FIG. 21 illustrates a side view of a fourth embodiment of the introducer apparatus, in accordance with aspects of the disclosure.

### DETAILED DESCRIPTION

Aspects will now be described more fully hereinafter with reference to the accompanying drawings in which example aspects are shown. Whenever possible, the same reference numerals are used throughout the drawings to refer to the same or like parts. However, this disclosure may be embodied in many different forms and should not be construed as limited to the aspects set forth herein.

As used herein, the term “about” means that amounts, sizes, formulations, parameters, and other quantities and characteristics are not, and need not be, exact, but may be approximate and/or larger or smaller, as desired, reflecting tolerances, conversion factors, rounding off, measurement error and the like, and other factors known to those of skill in the art.

Ranges can be expressed herein as from “about” one value, and/or to “about” another value. When such a range is expressed, aspects include from the one value to the other value. Similarly, when values are expressed as approximations by use of the antecedent “about,” it will be understood that the value forms another aspect. It will be further understood that the endpoints of each of the ranges are significant both in relation to the other endpoint, and independently of the other endpoint.

Directional terms as used herein-for example up, down, right, left, front, back, top, bottom, upper, lower, etc.-are made only with reference to the figures as drawn and are not intended to imply absolute orientation.

Unless otherwise expressly stated, it is in no way intended that any methods set forth herein be construed as requiring that its steps be performed in a specific order, nor that with any apparatus, specific orientations be required. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred in any respect. This holds for any possible non-express basis for interpretation, including matters of logic relative to arrangement of steps, operational flow, order of components, or orientation of components; plain meaning derived from grammatical organization or punctuation, and; the number or type of aspects described in the specification.

As used herein, the singular forms “a,” “an,” and “the” include plural references unless the context clearly dictates otherwise. Thus, for example, reference to “a” component includes aspects having two or more such components, unless the context clearly indicates otherwise.

The word “exemplary,” “example,” or various forms thereof are used herein to mean serving as an example, instance, or illustration. Any aspect or design described herein as “exemplary” or as an “example” should not be construed as preferred or advantageous over other aspects or designs. Furthermore, examples are provided solely for purposes of clarity and understanding and are not meant to limit or restrict the disclosed subject matter or relevant portions of this disclosure in any manner. It can be appreciated that a myriad of additional or alternate examples of varying scope could have been presented but have been omitted for purposes of brevity.

As used herein, the terms “comprising,” “including,” and variations thereof shall be construed as synonymous and open-ended, unless otherwise indicated. A list of elements following the transitional phrases comprising or including is a non-exclusive list, such that elements in addition to those specifically recited in the list may also be present.

The terms “substantial,” “substantially,” and variations thereof as used herein are intended to represent that a described feature is equal or approximately equal to a value or description. For example, a “substantially planar” surface is intended to denote a surface that is planar or approximately planar. Moreover, “substantially” is intended to denote that two values are equal or approximately equal. The term “substantially” may denote values within about 10% of each other, for example, within about 5% of each other, or within about 2% of each other.

Modifications may be made to the instant disclosure without departing from the scope or spirit of the claimed subject matter. Unless specified otherwise, “first,” “second,” or the like are not intended to imply a temporal aspect, a spatial aspect, an ordering, etc. Rather, such terms are merely used as identifiers, names, etc. for features, elements, items, etc. For example, a first end and a second end generally correspond to end A and end B or two different ends.

Unless otherwise indicated, the terms “distal” and “proximal” are used in the following description with respect to a position or direction relative to the treating clinician. “Distal” and “distally” are positions distant from or in a direction away from the clinician, and “proximal” and “proximally” are positions near or in a direction toward the clinician. In addition, the term “self-expanding” may be used in the following description with reference to one or more valve or stent structures of the prostheses hereof and is intended to convey that the structures are shaped or formed from a material that can be provided with a mechanical memory to return the structure from a compressed or constricted delivery configuration to an expanded deployed configuration or vice versa. Non-exhaustive exemplary self-expanding materials include stainless steel, a pseudo-elastic metal such as a nickel titanium alloy or nitinol, various polymers, or a so-called super alloy, which may have a hub metal of nickel, cobalt, chromium, or other metal. Mechanical memory may be imparted to a wire or stent structure by thermal treatment to achieve a spring temper in stainless steel, for example, or to set a shape memory in a susceptible metal alloy, such as nitinol. Various polymers that can be made to have shape memory characteristics may also be suitable for use in aspects hereof to include polymers such as polynorborene, trans-polyisoprene, styrene-butadiene, and polyurethane. As well poly L-D lactic copolymer, oligo caprylactone copolymer and poly cyclo-octine can be used separately or in conjunction with other shape memory polymers.

Diseases associated with heart valves, such as those caused by damage or a defect, can include stenosis and valvular insufficiency or regurgitation. For example, valvular stenosis causes the valve to become narrowed and hardened which can prevent blood flow to a downstream heart chamber from occurring at the proper flow rate and may cause the heart to work harder to pump the blood through the diseased valve. Valvular insufficiency or regurgitation occurs when the valve does not close completely, allowing blood to flow backwards, thereby causing the heart to be less efficient. A diseased or damaged valve, which can be congenital, age-related, drug-induced, or in some instances, caused by infection, can result in an enlarged, thickened heart that loses elasticity and efficiency. Some symptoms of heart valve diseases can include weakness, shortness of breath, dizziness, fainting, palpitations, anemia and edema, and blood clots which can increase the likelihood of stroke or pulmonary embolism. Symptoms can often be severe enough to be debilitating and/or life threatening.

Heart valve prostheses have been developed for repair and replacement of diseased and/or damaged heart valves. Such heart valve prostheses can be percutaneously delivered and deployed at the site of the diseased heart valve through catheter-hub delivery systems. Such heart valve prostheses generally include a frame or stent and a prosthetic valve mounted within the frame. Such heart valve prostheses are delivered in a radially compressed or crimped configuration so that the heart valve prosthesis can be advanced through the patient's vasculature. Once positioned at the treatment site, the heart valve prosthesis is expanded to engage tissue at the diseased heart valve region to, for instance, hold the heart valve prosthesis in position.

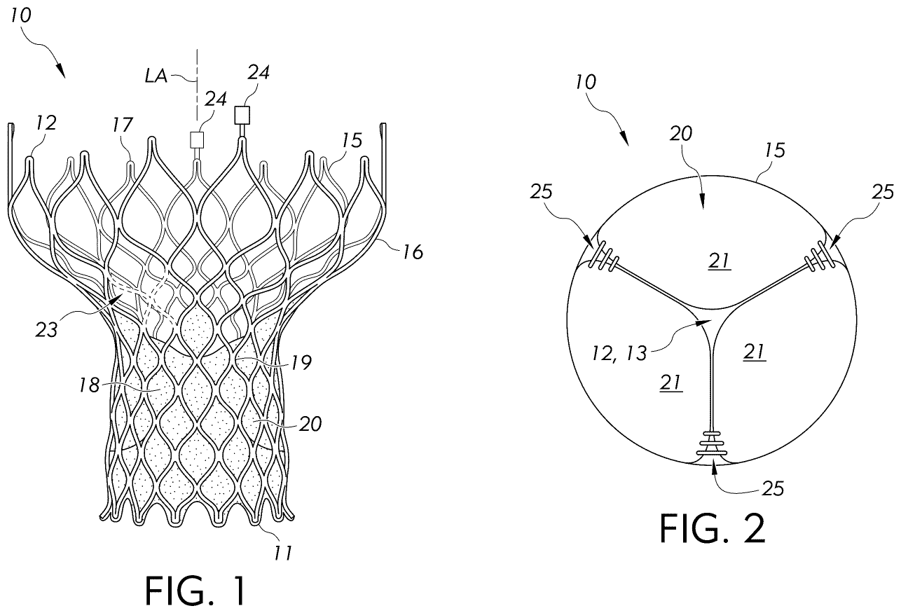

FIGS. 1 and 2 illustrate an example transcatheter heart valve prosthesis 10. The delivery assemblies described herein may be used with the transcatheter heart valve prosthesis 10 and/or other transcatheter heart valve prostheses. The transcatheter heart valve prosthesis 10 is illustrated to facilitate description of the disclosure. The following description of the transcatheter heart valve prosthesis 10 is merely exemplary in nature and is not intended to limit the invention or the application and uses of the invention.

FIGS. 1 and 2 illustrate a side view and a top/end view, respectively, of the transcatheter heart valve prosthesis 10. The transcatheter heart valve prosthesis 10 includes a radially-expandable frame or stent 15 and a prosthetic valve 20. The frame 15 of the transcatheter heart valve prosthesis 10 supports the prosthetic valve 20 within an interior of the frame 15. In the example transcatheter heart valve prosthesis 10 shown in FIGS. 1 and 2, the frame 15 is self-expandable. However, this is not meant to be limiting, and the frame 15 can be balloon-expandable or mechanically expandable in other embodiments. In some embodiments, the transcatheter heart valve prosthesis 10 may be delivered to and implanted at a treatment site within a patient to replace any of an aortic valve, a pulmonic valve, a mitral valve, and a tricuspid valve. The valve to be replaced may be a native valve or a previously-implanted prosthetic valve, such as a failed surgical replacement valve or a failed transcatheter valve.

The prosthetic valve 20 includes at least one leaflet 21 disposed within and secured to the frame 15. In the embodiment shown in FIGS. 1 and 2, the prosthetic valve 20 includes exactly three leaflets 21, as shown in FIG. 2. However, this is not meant to be limiting, as the prosthetic valve 20 may include more or fewer leaflets 21. The valve leaflets 21 open and close to regulate flow through the transcatheter heart valve prosthesis 10.

As shown in FIG. 1, the transcatheter heart valve prosthesis 10 includes an inflow end 11 and an outflow end 12. The prosthetic leaflets 21 are attached to the frame 15 at commissures 25 such that when pressure at the inflow end 11 exceeds pressure at the outflow end 12, the prosthetic leaflets 21 open to allow blood flow through the heart valve prosthesis 10 from the inflow end 11 to the outflow end 12. When the pressure at the outflow end 12 exceeds pressure at the inflow end 11, the prosthetic leaflets 21 close to prevent blood flow from the outflow end 12 to the inflow end 11. Accordingly, the at least one leaflet (e.g., the prosthetic leaflets 21) can be attached to the plurality of struts 16, for example, by being directly attached to the plurality of struts 16 at the commissures 25, or by being indirectly attached to the plurality of struts 16, for example, by being attached to a skirt, a commissure bracket, or other structure (e.g., mechanical actuator) that is attached to the plurality of struts 16. In aspects, the heart valve prosthesis 10 can comprise one or more attachment members 24 (e.g., paddles) positioned at an end, for example, the outflow end 12. The attachment members 24 can be received within pockets of a spindle 38 (e.g., illustrated in FIG. 4), such that the spindle 38 and the attachment members 24 can interact to facilitate loading of the transcatheter heart valve prosthesis 10 and, in aspects, allow for possible recapture of the transcatheter heart valve prosthesis 10 during the deployment process.

The frame 15 of the transcatheter heart valve prosthesis 10 further includes a plurality of struts 16 that are arranged to form a plurality of openings or cells 18 arranged circumferentially around a longitudinal axis LA of the transcatheter heart valve prosthesis 10 and longitudinally to form a tubular structure defining a central lumen of the transcatheter heart valve prosthesis 10. For example, the frame 15 can extend along the longitudinal axis LA between the inflow end 11 and the outflow end 12. The frame 15 is configured to secure the prosthetic valve 20 within the central lumen of the frame 15 and to secure the transcatheter heart valve prosthesis 10 in place in the vasculature of the patient. The struts 16 are defined herein as the elongated wire segments of the frame 15. Struts 16 come together to form crowns 17 or nodes 19, as can be seen in FIG. 1. The frame 15 of the heart valve prosthesis 10 includes a plurality of cells 18 defined as the spaces between the plurality of crowns 17, the plurality of nodes 19, and the plurality of struts 16. The frame 15, and, thus, the plurality of struts 16, can be adjustable between a radially-collapsed position and a radially-expanded position.

In the example embodiment shown in FIG. 1, the plurality of cells 18 may be diamond-shaped. In the example embodiment shown, the plurality of cells include a plurality of first cells 18 and, in aspects, access cells (e.g., an access cell 23). In particular, the access cells may be larger than the first cells 18 and can provide access to one or more coronary arteries when the transcatheter heart valve prosthesis 10 is implanted in the patient. FIG. 1 illustrates an example of an access cell 23, with the struts 16 at the access cell 23 illustrated with dashed lines to show that the struts 16 may not be present at the access cell 23, thus allowing for the access cell 23 to be larger than the first cells 18. The access cells can have an enlarged area relative or compared to the first cells 18. In some embodiments the transcatheter heart valve prosthesis 10 may include an outer skirt extending circumferentially around an outer circumference of the stent 15 at or near the inflow end 11 to prevent paravalvular leakage of blood around the outside of the transcatheter heart valve prosthesis 10 once implanted in the patient.

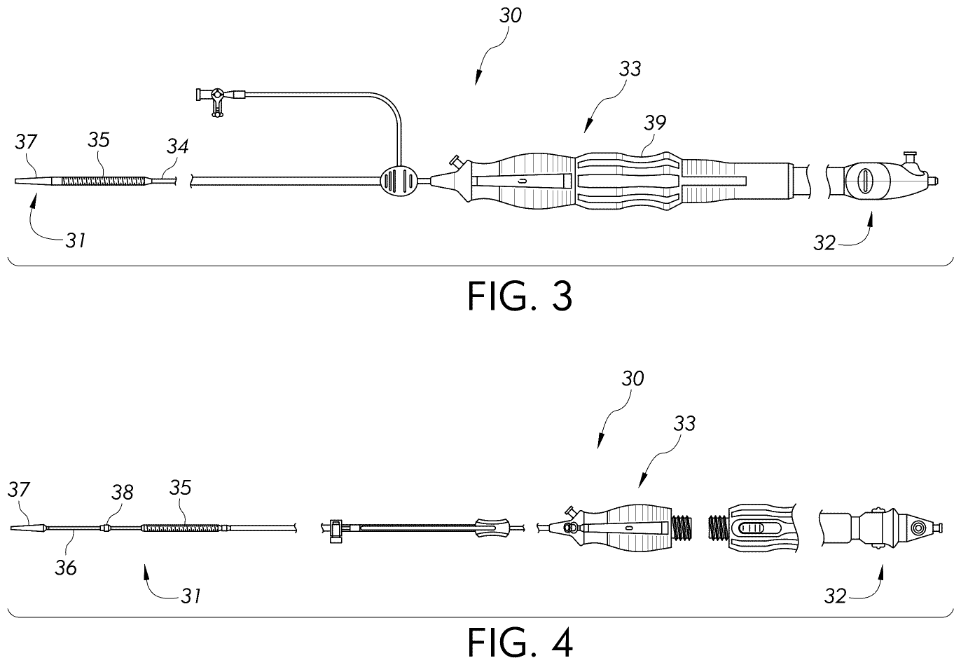

FIGS. 3 and 4 show schematically side views of a transcatheter heart valve delivery assembly 30 (e.g., “delivery assembly”) for delivering and deploying a transcatheter heart valve prosthesis (e.g., transcatheter heart valve prosthesis 10) according to embodiments hereof. One skilled in the art will realize that FIGS. 3 and 4 illustrate one example of a delivery assembly 30 and that components illustrated in FIGS. 3 and 4 may be removed and/or additional components may be added. The delivery assembly 30 includes a distal end 31, a proximal end 32, and a handle 33. The handle 33 enables a physician to manipulate a distal portion of the delivery assembly 30 and includes actuators for moving parts of the delivery assembly 30 relative to other parts. In the delivery assembly 30, an outer shaft 34 is coupled to an actuator 39 of the handle 33 for moving the outer shaft 34 relative to an inner shaft 36.

A distal portion of the outer shaft 34, referred to as a capsule 35, is configured to surround a transcatheter heart valve prosthesis (e.g., transcatheter heart valve prosthesis 10) during delivery to the treatment site (e.g., a native heart valve) and is retracted from the transcatheter heart valve prosthesis to expose the transcatheter heart valve prosthesis such that it self-expands (in self-expanding embodiments). In this way, the capsule 35 is in frictional engagement with the heart valve prosthesis 10. The inner shaft 36 can be coupled to the handle 33 (e.g., by being directly connected and in contact with the handle 33, or by being indirectly connected to the handle 33 with intermediate structures between the inner shaft 36 and the handle 33) and movement of the handle 33 can translate to movement of the inner shaft 36 and a distal tip or nosecone 37 coupled to a distal end of the inner shaft 36. The inner shaft 36 and distal tip or nosecone 37 may also be translated relative to the outer shaft 34 and the handle 33 via a tip retractor. In the embodiment shown, the inner shaft 36 includes a retainer or spindle 38 for receiving the paddles (e.g., attachment members 24) of the transcatheter heart valve prosthesis 10.

When the actuator 39 is actuated, the actuator 39 moves the outer shaft 34 and the capsule 35 relative to the inner shaft 36, as shown in FIG. 4. As known to those skilled in the art, when the delivery assembly 30 is in position such that the transcatheter heart valve prosthesis 10 is at the desired position at the treatment site in the patient's vasculature, the actuator 39 is actuated (e.g., rotated) to move the capsule 35 relative to the inner shaft 36 and the transcatheter heart valve prosthesis 10 disposed between the inner shaft 36 and the capsule 35, thereby enabling the transcatheter heart valve prosthesis 10 to deploy via self-expansion at the treatment site and release from the retainer 38, as shown in FIG. 4 (without showing the transcatheter heart valve prosthesis 10).

Minimally invasive percutaneous interventional procedures, including endovascular procedures, require access to the venous or arterial system. In general, it is desirable to make the smallest incision point with the shortest tissue contact time when entering the body. Small incisions and short tissue contact time generally lead to improved patient outcomes, less complications, and less trauma to the vessels or organs being accessed, as well as less trauma to the skin and tissue through which the access point is created. Access is required for various medical procedures that deliver or implant structural elements (such as heart valves, heart valve repair devices, occluders, grafts, electrical stimulators, leads, etc.) percutaneously. Some procedures employ relatively large devices that require relatively large sheaths to deliver the devices to the intended site within the body. With such procedures, access site trauma can occur, often resulting in vessel damage, excessive bleeding, increased case time, increased risk of infection, and increased hospitalization time. To reduce access trauma, physicians try to use the smallest devices possible and place the smallest sheath size. This can be problematic, however, if during the procedure the physician discovers a larger device is needed. This leads to a need to upsize the sheath, which is a lengthy procedure and leads to increased risk to the patient. Expandable sheaths can be expanded within the body and thus do not require removal to upsize.

Expandable sheath designs may be regionally or locally expansive to selectively and temporarily expand when the device is passing through a region of the sheath and to retract or recover when the device is not passing or has already passed through the sheath. Embodiments disclosed herein may be employed with an expandable introducer sheath that may solve these and other issues that contribute to vascular trauma. The expandable introducer sheath is described with respect to percutaneous access for transcatheter heart valve repair or replacement, and it should be understood that one or more features of the expandable introducer sheath may be employed alone or in combination for other medical procedures requiring percutaneous access, including but not limited to placement of stents, angioplasty, removal of arterial or venous calcification, and pre-dilatation or post-dilatation.

Various embodiments disclosed herein may include an introducer sheath that has a selectively expandable diameter to allow for the passage of a relatively larger device therethrough and further is configured to return to its original diameter upon passage of the device. The various embodiments may reduce damage to surrounding tissues by reducing contact with those tissues and by eliminating the need to exchange sheaths of different sizes. As a result, these embodiments can reduce procedure time, vascular trauma, bleeding, and the resulting risk of infection and other complications.

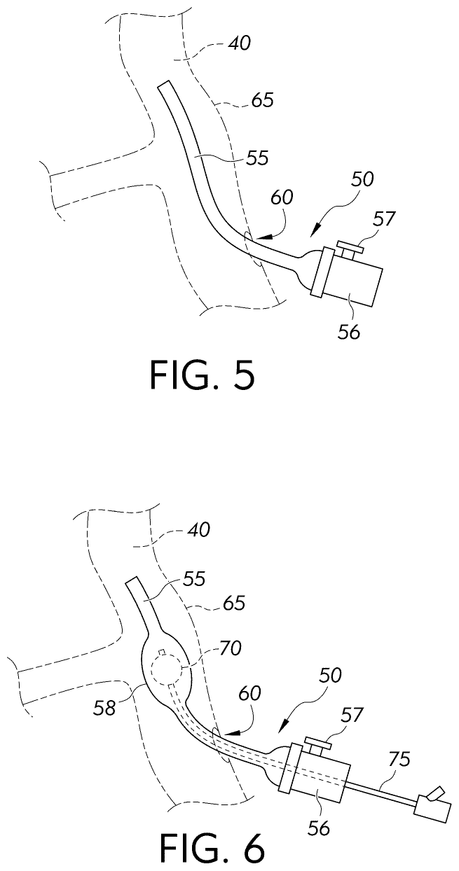

FIGS. 5 and 6 depict one embodiment of an introducer apparatus 50 positioned through an incision 60 in the skin 65 of a patient and into a vessel 40 of a patient. The introducer apparatus 50 has a tubular sheath 55 and a proximal hub 56 with a hemostatic seal and a luer lock 57. FIG. 5 shows the sheath 55 of the introducer apparatus 50 positioned in the vessel 40 in its normal, unexpanded state, while FIG. 6 shows the introducer apparatus 50 positioned in the vessel 40 with a delivery device 75 delivering another device 70 that is being advanced through the introducer apparatus 50 such that the tubular sheath 55 expands or deforms at the location where the device 70 is passing through. The sheath 55 expands at expanded region 58 when the device 70 passes through and then retracts or recovers to its original diameter after the device 70 moves past or is removed from the sheath 55. Thus, the tubular sheath 55 is configured to be expandable and retractable.

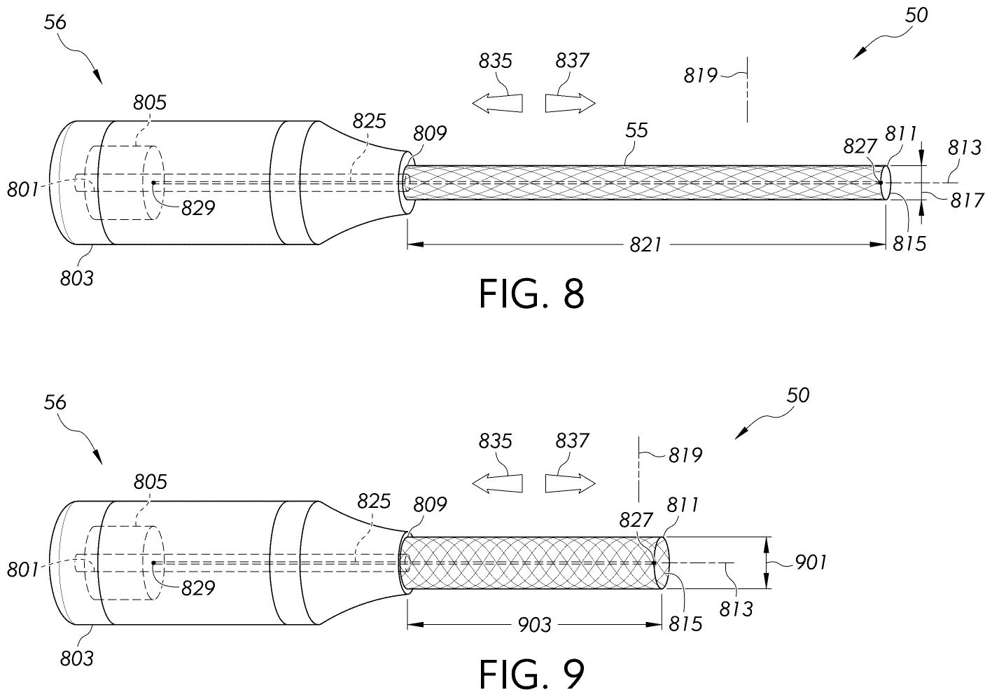

In certain embodiments, the expandability of the sheath 55 (and any sheath described according to any embodiment set forth herein) is achieved via the elasticity of the sheath 55, which can result in the sheath 55 being either self-expandable or self-expanding or mechanically expandable or mechanically expanding. For purposes of this application, self-expandable means that the sheath 55 is configured to expand to a predetermined or nominal diameter automatically (without any type of actuation, mechanical or otherwise). Further, for purposes of this application, mechanically expandable means that the sheath 55 is configured to expand when a positionable medical device is positioned through the sheath 55. That is, the device itself that is being passed through the sheath 55 causes the expansion of the sheath 55, as depicted in FIG. 6. Alternatively, and as described below relative to FIGS. 8-21, the expandable characteristics of the sheath 55 can be caused by one or more wires that move the sheath 55 between an expanded position with a smaller diameter and a retracted position with a larger diameter.

After passage of the device, the sheath 55 is configured to be contractable, retractable, or recoverable to its original, unexpanded state as depicted in FIG. 5. The retractability can be, in certain embodiments, achieved by the elasticity of the sheath 55, which can result in the sheath 55 being either self-retractable or self-retracting, self-recoverable, or self-contractable, or mechanically retractable or mechanically retracting, mechanically recoverable, or mechanically contractable. For purposes of this application, self-retractable means that the sheath 55 is configured to retract to a predetermined or nominal diameter automatically (without any type of actuation, mechanical or otherwise). Further, for purposes of this application, mechanically retractable means that the sheath 55 is configured to retract when a device or component is used to cause the sheath 55 to retract or recover. Alternatively, the retractable characteristics of the sheath 55 can be caused by something other than elasticity.

For purposes of this application, any device that can be positioned through an introducer sheath according to any embodiment disclosed or contemplated herein can be referred to as a positionable medical device or insertable medical device. Such devices include guidewires, dilators, delivery devices (for delivery and/or placement of structural elements such as heart valves, heart valve repair devices, occluders, grafts, electrical stimulators, leads, etc.), guide catheters, guiding sheaths, diagnostic catheters, stent delivery systems, balloon catheters, and other known vascular devices. Other devices can include non-vascular devices such as scopes and other common surgical instruments. Further, the introducer sheath is configured to receive tissues or organs. Thus, as one non-limiting example, the introducer sheath 55 is described as being an expandable introducer sheath 55 for introduction of a delivery assembly 30 including a transcatheter heart valve prosthesis 10.

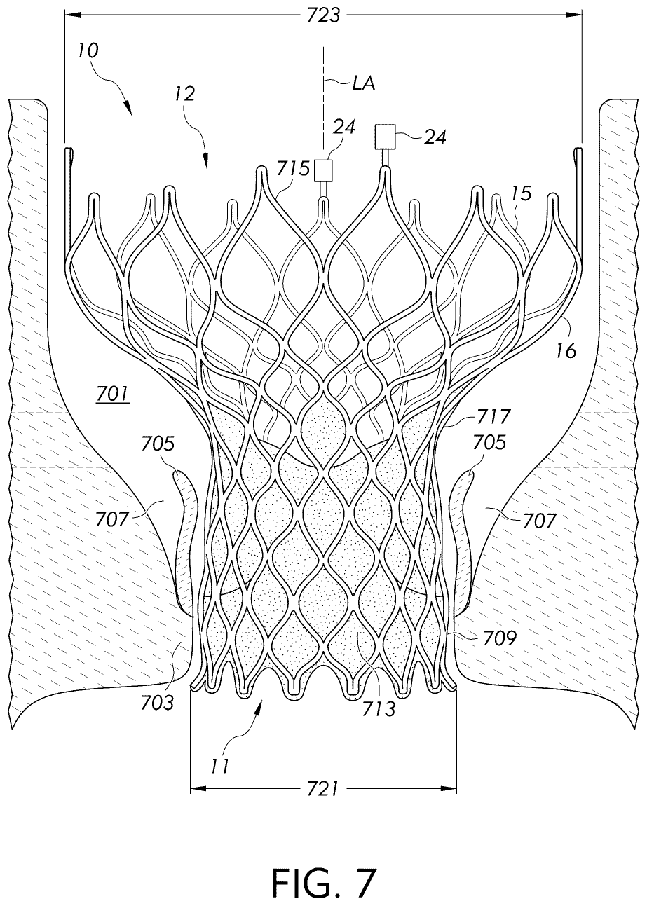

FIG. 7 illustrates the heart valve prosthesis 10 at a treatment site 701 within a patient's vasculature. In aspects, the treatment site 701 can comprise a location of a native aortic annulus (hereinafter “annulus”) 703 of a native heart valve, for example, the annulus of a patient's left ventricle. The treatment site 701 can comprise one or more native valve leaflets 705 and corresponding native sinuses 707. In some instances, paravalvular leakage can occur when blood travels through a gap 709 around the outside of the transcatheter heart valve prosthesis 10, with the gap 709 formed between the transcatheter heart valve prosthesis 10 and the annulus 703. To avoid paravalvular leakage, the heart valve prosthesis 10 can be radially expanded such that an outer radial surface of the heart valve prosthesis 10 can contact the annulus 703 and/or the native valve leaflets 705, thus reducing or eliminating the gap 709 and causing the blood to flow through the central lumen 13 of the heart valve prosthesis 10. The frame 15 of the heart valve prosthesis 10 can comprise an asymmetric hourglass shape with a first section 713 at the inflow end 11, a second section 715 at the outflow end 12, and a waist section 717 positioned between the first section 713 and the second section 715. In aspects, the first section 713 can comprise a first diameter 721 and the second section 715 can comprise a second diameter 723, with the second diameter 723 greater than the first diameter 721. Additionally, in some embodiments the transcatheter heart valve prosthesis 10 may include an outer skirt extending circumferentially around an outer circumference of the frame 15 at or near the inflow end 11 to prevent paravalvular leakage of blood around the outside of the transcatheter heart valve prosthesis 10 once implanted in the patient. Thus, features of the disclosure may be employed alone or in combination with a heart valve prosthesis 10 having an outer skirt or other external sealing member (not shown) or a heart valve prosthesis 10 having no outer skirt.

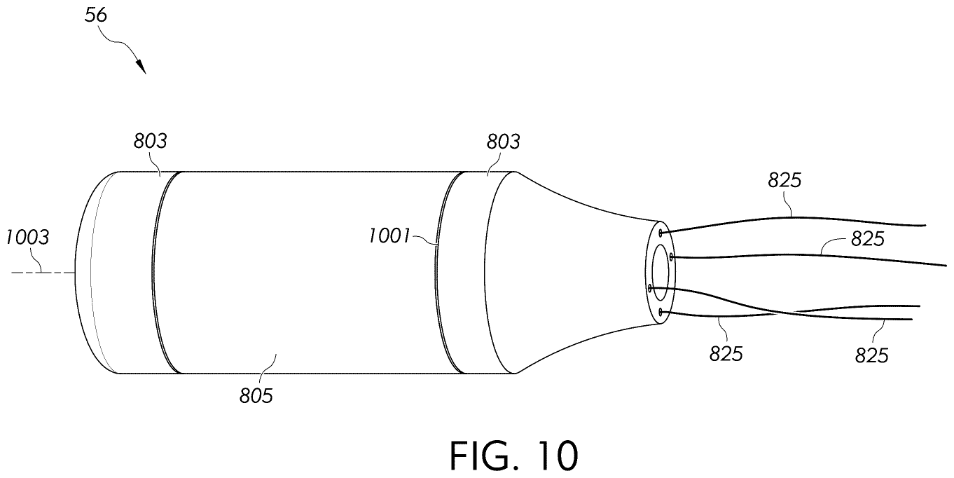

FIGS. 8-9 illustrate a side view of the introducer apparatus 50 comprising the sheath 55 and the hub 56. The hub 56 can comprise a central passage 801 that is substantially hollow and through which one or more structures can pass. The hub 56 can comprise a body portion 803 and an engagement member 805. The body portion 803 can circumferentially surround the central passage 801 such that one or more walls of the body portion 803 can at least partially define the central passage 801. Portions of the hub 56, for example, the central passage 801, the body portion 803, and the engagement member 805, are illustrated schematically in FIGS. 8-9 due to the central passage 801, the body portion 803, and the engagement member 805 comprising several different embodiments illustrated in FIGS. 10-21. As such, FIG. 8 generally illustrates a position of the sheath 55 relative to the hub 56, wherein the sheath 55 is in an expanded position. FIG. 9 generally illustrates movement of the sheath 55 relative to the hub 56 such that the sheath 55 is in a retracted position. However, the position and structure of the engagement member 805 relative to the body portion 803 is not intended to be limited to the examples illustrated in FIGS. 8-9, as the engagement member 805 is illustrated schematically. Rather, the embodiments of FIGS. 10-21 illustrate other examples of the position and structure of the engagement member 805.

The sheath 55 can extend between a proximal sheath end 809 and a distal sheath end 811 along a central axis 813. The sheath 55 can be attached to the hub 56, for example, with the proximal sheath end 809 attached to the hub 56. The sheath 55 can comprise a lumen 815 (e.g., elongated passageway) that extends along the length of the sheath 55 between the proximal sheath end 809 and the distal sheath end 811. In this way, the lumen 815 may be in communication with the central passage 801. By being in communication, the lumen 815 and the central passage 801 can be oriented relative to one another such that an object (e.g., the delivery device 75, the heart valve prosthesis 10, etc.) can pass from the central passage 801 and into the lumen 815 and vice versa. Though not required, in aspects, the lumen 815 and the central passage 801 can be positioned in an end-to-end orientation.

The sheath 55 can comprise a first cross-sectional dimension 817 along a plane 819 that is perpendicular to the central axis 813. In aspects, the sheath 55 can comprise a substantially circular cross-sectional shape, such that the first cross-sectional dimension 817 comprises a first diameter of the sheath 55. The sheath 55 can comprise a first length 821 that is measured along the central axis 813 between the hub 56 and the distal sheath end 811. In aspects, the sheath 55 can be biased toward the first cross-sectional dimension 817 and the first length 821 such that, in the absence of forces (e.g., compressive, tensile, etc.) acting upon the sheath 55, the sheath 55 comprises the first cross-sectional dimension 817 and the first length 821. However, as described herein, the first cross-sectional dimension 817 and the first length 821 can be changed due to forces acting upon the sheath 55. Methods can therefore comprise providing the sheath 55 attached to the hub 56, with the sheath 55 comprising the first cross-sectional dimension 817 along the plane 819 that is perpendicular to the central axis 813.

The introducer apparatus 50 can comprise a plurality of wires 825 extending between a first end 827 and an opposing second end 829. The first end 827 can be attached to the distal sheath end 811 of the sheath 55, and the second end 829 can be attached to the hub 56, for example, to the engagement member 805. In aspects, the plurality of wires 825 may extend through the lumen 815 of the sheath 55 between the engagement member 805 and the distal sheath end 811. The plurality of wires 825 can comprise several types of wires. For example, in aspects, the plurality of wires 825 can comprise suture wires that can provide a pulling force (e.g., in a proximal direction 835) but have limited to no ability to provide a pushing force (e.g., in a distal direction 837). Alternatively, in aspects, the plurality of wires 825 can comprise wires of a higher stiffness that can provide both a pulling force and a pushing force. In aspects, when the plurality of wires 825 comprise a higher stiffness to provide both the pulling force and a pushing force, the sheath 55 may be biased (e.g., in a resting state in the absence of forces applied to the sheath 55) toward the retracted position illustrated in FIG. 9, such that the plurality of wires 825 can apply a pushing force to the sheath 55 to move the sheath 55 from the retracted position illustrated in FIG. 9 to the expanded position illustrated in FIG. 8. Indeed, in aspects, the sheath 55 can comprise three possible states or diameters: (1) a first, or neutral state/position, wherein the sheath 55 has a diameter that is greater than the diameter 817 but less than the diameter 901; (2) a second, or expanded, state/position, wherein the sheath 55 has the diameter 817 of FIGS. 8; and (3) a third, or retracted, state/position, wherein the sheath 55 has the diameter 901 of FIG. 9. In such an example, the sheath 55 can be moved between the three states/diameters by applying a pushing force or pulling force via the plurality of wires 825. In this way, the sheath 55 may be biased (e.g., in a resting state) toward the first, or neutral state/position, and may be moved to the second, or expanded, state/position by applying a pushing force to the sheath 55 via the wires 825, and, conversely, may be moved to the third, or retracted, state/position by applying a pulling force to the sheath 55 via the wires 825.

The engagement member 805 can move the plurality of wires 825 and the distal sheath end 811 between an extended position (e.g., illustrated in FIG. 8), in which the sheath 55 comprises the first cross-sectional dimension 817 and the first length 821, and a retracted position (e.g., illustrated in FIG. 9), in which the sheath 55 comprises a second cross-sectional dimension 901 and a second length 903. As described in the embodiments of FIGS. 10-21, the engagement member 805 can move relative to the body portion 803, for example, by rotating relative to the body portion 803, moving axially relative to the body portion 803, etc. which can cause the plurality of wires 825 to move in the proximal direction 835 or the distal direction 837.

Referring to FIG. 9, the engagement member 805 can move relative to the body portion 803 to cause the plurality of wires 825 to move in the proximal direction 835, which can also cause the distal sheath end 811 to move in the proximal direction 835. Methods can therefore comprise moving the plurality of wires 825 that are attached to the distal sheath end 811 of the sheath 55 in the proximal direction 835 toward the hub 56. The movement of the engagement member 805 can increase the tension of the plurality of wires 825 and cause the plurality of wires 825 to move in the proximal direction 835. Due to the attachment of the first end 827 of the plurality of wires 825 to the distal sheath end 811, the distal sheath end 811 can move in the proximal direction 835, which can increase the compression of the sheath 55. This increase in compression of the sheath 55 can cause an increase of cross-sectional dimension (e.g., diameter) of the sheath 55, from the first cross-sectional dimension 817 to the second cross-sectional dimension 901. The second cross-sectional dimension 901 is greater than the first cross-sectional dimension 817, such that when the distal sheath end 811 is moved in the proximal direction 835, the sheath 55 can increase in cross-sectional size or diameter. The second length 903 is less than the first length 821, such that when the distal sheath end 811 is moved in the proximal direction 835, the length of the sheath 55 can decrease.

By controlling the cross-sectional size of the sheath 55 (e.g., via the plurality of wires 825), the resistance forces that act upon passing structures (e.g., delivery device 75, prosthesis 10, etc.) that pass through the lumen 815 of the sheath 55 can be reduced. That is, when passing structures pass through the lumen 815, contact between the passing structures and the sheath 55 may tend to increase tension to be applied to the sheath 55, which causes constriction of the sheath 55 and increases friction between the sheath 55 and the passing structures. However, by increasing the cross-sectional size of the sheath 55 by applying the pulling force to the distal sheath end 811, the likelihood of contact between the passing structures and an inner radial surface of the sheath 55 is decreased and/or the amount of friction between the passing structures and the inner radial surface of the sheath 55 is decreased. In the absence of tension applied to the distal sheath end 811 by the plurality of wires 825, the sheath 55 can revert back to the position and dimensions illustrated in FIG. 8, thus not restricting blood flow through the vessel 40. In addition, or in the alternative, the plurality of wires 825 can comprise a stiffer material that can apply a pushing force (e.g., in the distal direction 837) to facilitate movement of the sheath 55 to the position and dimensions illustrated in FIG. 8. Accordingly, when no passing structures are within the lumen 815, the sheath 55 may be at the smaller, first cross-sectional dimension 817, but when passing structures are within the lumen 815, the sheath 55 may expand to the larger, second cross-sectional dimension 817. In aspects, the sheath 55 can comprise several materials, such as a Nitinol mesh. In aspects, the first cross-sectional dimension 817 may be within a range from about 12 Fr (e.g., French gauge) to about 20 Fr, and the second cross-sectional dimension 901 may be within a range from about 20 Fr to about 26 Fr. In one example, the first cross-sectional dimension 817 is about 18 Fr and the second cross-sectional dimension 901 is about 24 Fr. However, these dimensions are merely examples, and other possible dimensions are envisioned.

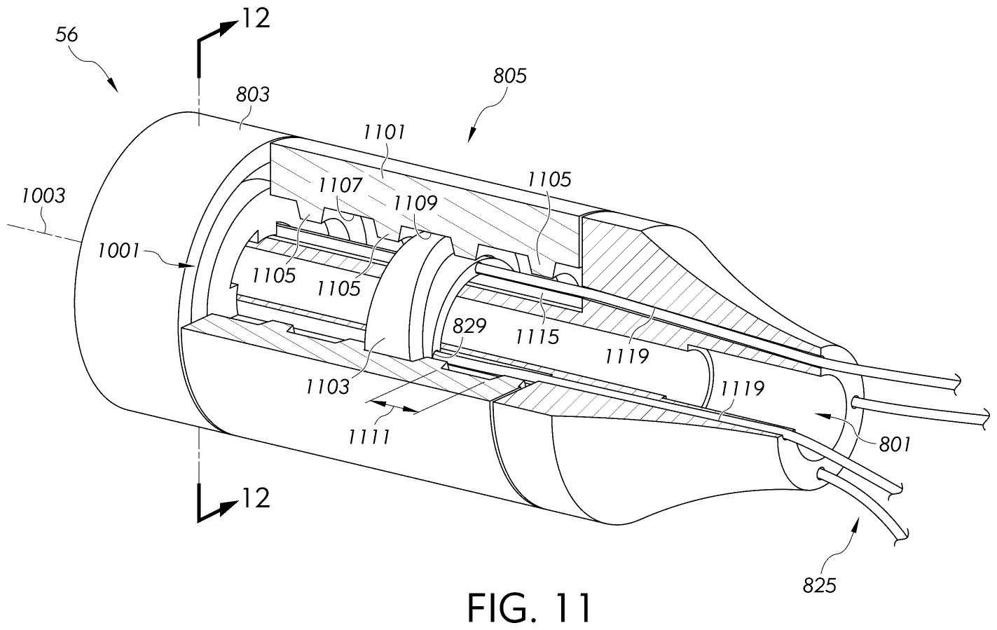

FIGS. 10-13 illustrate an embodiment of the hub 56 comprising the body portion 803 and the engagement member 805. For example, FIG. 10 illustrates a side view of the hub 56 in engagement with the plurality of wires 825. For purposes of illustration, the sheath 55 is not illustrated in FIGS. 10-11 so as to not obscure view of the plurality of wires 825. However, in operation, the sheath 55 is attached to the hub 56 as illustrated in FIGS. 8-9 and FIGS. 12-13. As illustrated in FIG. 10, the engagement member 805 can be positioned within a recess 1001 of the hub 56 that is bordered on opposing axial sides by the body portion 803. The engagement member 805 can therefore be attached to the body portion 803, with the engagement member 805 configured to move relative to the body portion 803. In this way, by being attached, the engagement member 805 can be connected to the body portion 803 (e.g., by being received within the recess 1001) while not being limited to a single fixed position relative to the body portion 803. Rather, in aspects, the engagement member 805 can rotate relative to the body portion 803, for example, about a hub axis 1003 along which the hub 56 extends. The body portion 803 and the engagement member 805 can circumferentially surround the hub axis 1003 and the central passage 801 while extending coaxially with one another.

FIG. 11 illustrates a partial sectional view of the hub 56 with a segment of the body portion 803 and the engagement member 805 removed in order to illustrate additional features of the hub 56, wherein the segment is parallel to the hub axis 1003. In aspects, the engagement member 805 can comprise a plurality of portions that can move relative to the body portion 803. For example, the engagement member 805 can comprise a first engagement portion 1101 and a second engagement portion 1103. The first engagement portion 1101 may circumferentially surround the second engagement portion 1103, such that the second engagement portion 1103 is received within the first engagement portion 1101. The first engagement portion 1101 is therefore positioned within the recess 1001, with the first engagement portion 1101 comprising a length (e.g., along the hub axis 1003) that substantially matches, or is slightly less than, the length of the recess 1001. In aspects, the body portion 803 and the first engagement portion 1101 can comprise substantially matching cross-sectional sizes (e.g., diameters in FIGS. 10-11 due to the substantially cylindrical shapes of the body portion 803 and the first engagement portion 1101), though, differing cross-sectional sizes are possible. Accordingly, the first engagement portion 1101 is rotatable relative to the body portion 803, with the first engagement portion 1101 circumferentially surrounding the central passage 801.

In aspects, the first engagement portion 1101 can comprise one or more threads 1105 positioned on an inner radial side 1107 of the first engagement portion 1101. The threads 1105 can wind helically around the hub axis 1003 on the inner radial side 1107 while extending at least partially along a length of the first engagement portion 1101. The threads 1105 can be spaced axially apart along the hub axis 1003 to define a channel 1109 that winds helically around the hub axis 1003 between threads 1105. The threads 1105 can project from an inner radial surface of the first engagement portion 1101 radially inwardly toward the hub axis 1003. In this way, the inner radial surface is spaced a non-constant radial distance from the hub axis 1003 along a length of the first engagement portion 1101 due to the presence of the threads 1105 and the channel 1109. The threads 1105 can facilitate the conversion of rotational movement (e.g., from rotation of the first engagement portion 1101) to linear movement (e.g., linear movement of the second engagement portion 1103).

The engagement member 805 can comprise the second engagement portion 1103 that is received within the first engagement portion 1101. The second engagement portion 1103 can move axially relative to the first engagement portion 1101. For example, in aspects, the second engagement portion 1103 can comprise a length 1111 that is less than the distance between neighboring threads 1105, such that the second engagement portion 1103 can be positioned within the channel 1109. In aspects, the second engagement portion 1103 can be positioned adjacent to one or more detent structures 1115 of the body portion 803 that limit rotation of the second engagement portion 1103 about the hub axis 1003. For example, the one or more detent structures 1115 can comprise a ledge, outcropping, protuberance, extension, or the like that is adjacent to, and in contact with, circumferential ends of the second engagement portion 1103. In aspects, a first circumferential end of the second engagement portion 1103 may be in contact with one detent structure 1115, while an opposing second circumferential end of the second engagement portion 1103 may be in contact with another detent structure 1115. In this way, the second engagement portion 1103 is limited from rotating due to being positioned circumferentially between detent structures 1115. However, in aspects, other ways of limiting rotation of the second engagement portion 1103 can be provided, such that the hub 56 is not limited to the detent structures 1115.

In aspects, the second end 829 of the plurality of wires 825 can be attached to the second engagement portion 1103 of the engagement member 805, with the plurality of wires 825 extending through the central passage 801 of the hub 56. The second end 829 of the plurality of wires 825 can be attached in any number of ways, for example, by adhesives, passing through channels of the second engagement portion 1103, knots or other fastening mechanisms, etc. By being attached, movement of the second engagement portion 1103 can cause corresponding movement of the plurality of wires 825. Though not required, in aspects, the central passage 801 of the body portion 803 can comprise one or more discrete wire openings 1119 through which the plurality of wires 825 can extend, with the one or more wire openings 1119 extending along the central axis 813 and the hub axis 1003.

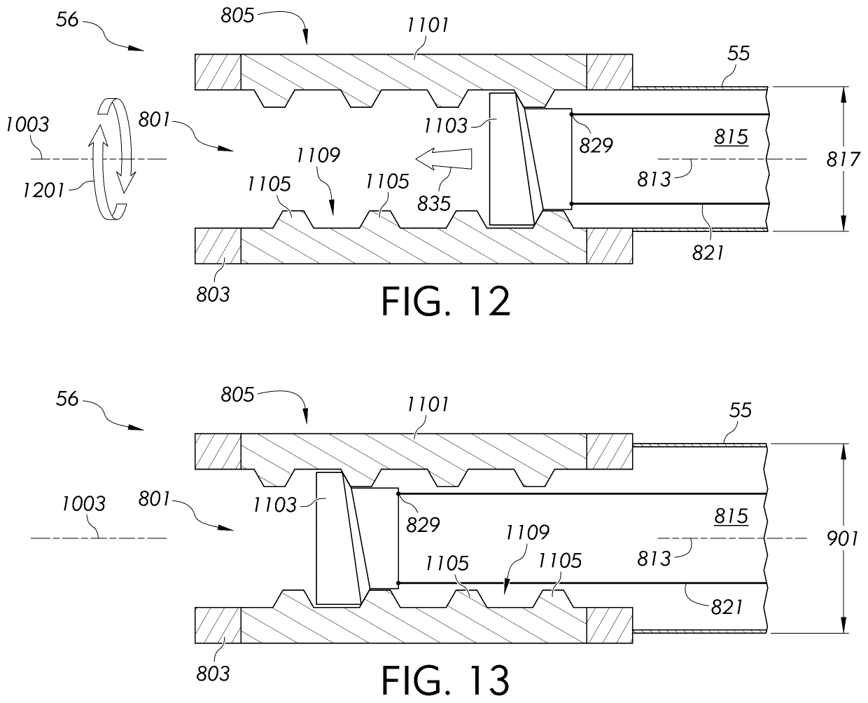

FIGS. 12-13 illustrate a cross-sectional view of the hub 56 along lines 12-12 of FIG. 11. For example, FIG. 12 illustrates a position of the second engagement portion 1103 when the second engagement portion 1103 and the sheath 55 are in an extended position, which corresponds to the extended position illustrated in FIG. 8. As illustrated in FIG. 12, the second engagement portion 1103 is received within the channel 1109 between threads 1105. Methods can comprise moving the plurality of wires 825 by rotating (e.g., illustrated schematically with arrowheads 1201) the first engagement portion 1101 relative to the body portion 803 of the hub 56 such that the second engagement portion 1103, which is received within the first engagement portion 1101, moves axially relative to the first engagement portion 1101. For example, as the first engagement portion 1101 rotates, the second engagement portion 1103 can move axially in a movement direction (e.g., the proximal direction 835) along the hub axis 1003. The detent structures 1115 of FIG. 11 can limit the second engagement portion 1103 from rotating, such that the second engagement portion 1103 is limited to moving axially in response to rotation of the first engagement portion 1101.

Referring to FIG. 13, the first engagement portion 1101 can continue to rotate 1201 at least until the second engagement portion 1103 and the sheath 55 are in a retracted position, which corresponds to the retracted position illustrated in FIG. 9. As illustrated in FIG. 13, rotation of the first engagement portion 1101 can move the second engagement portion 1103 axially in the proximal direction 835 along the hub axis 1003. This proximal movement of the second engagement portion 1103 can likewise cause the plurality of wires 825 to move in the proximal direction 835. Accordingly, axial movement (e.g., along the axes 813, 1003) of the second engagement portion 1103 can cause the plurality of wires 825 and the distal sheath end 811 of the sheath 55 to move in the proximal direction 835. As such, the sheath 55 can move from the extended position, comprising the first cross-sectional dimension 817 and the first length 821 (e.g., illustrated in FIG. 8 and FIG. 12), to the retracted position, comprising the second cross-sectional dimension 901 and the second length 903 (e.g., illustrated in FIG. 9 and FIG. 13).

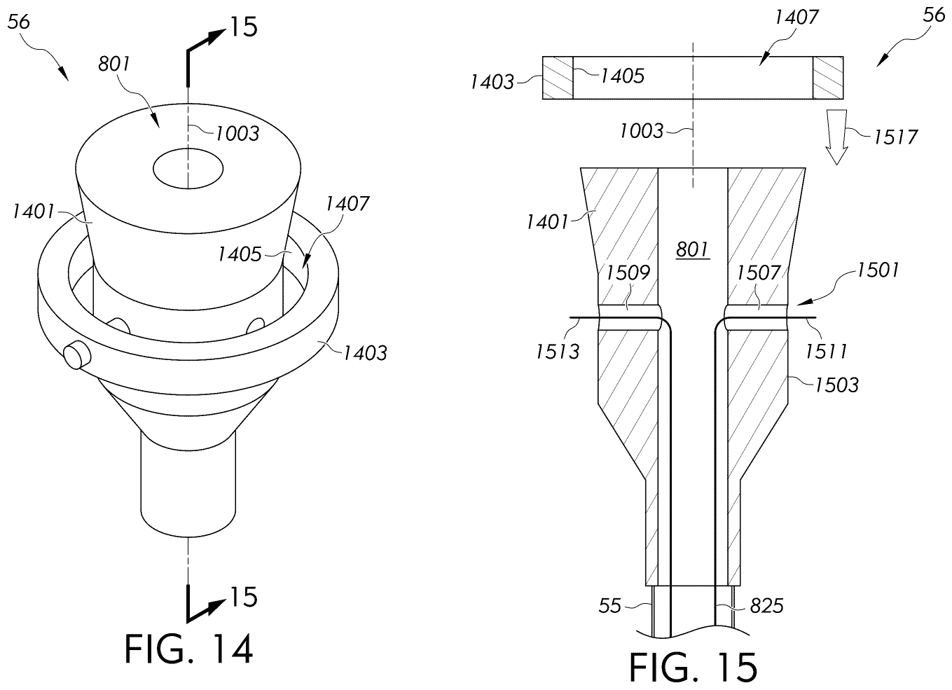

FIGS. 14-18 illustrate another embodiment of the hub 56 comprising a body portion 1401 and an engagement member 1403. For purposes of illustration, the sheath 55 is not illustrated in FIG. 14 so as to not obscure view of the plurality of wires 825. However, in operation, the sheath 55 is attached to the hub 56 as illustrated in FIGS. 8-9 and FIGS. 15-16. As illustrated in FIG. 14, the engagement member 1403 can be positioned to circumferentially surround the body portion 1401. The engagement member 1403 can be attached to the body portion 1401, with the engagement member 1403 configured to move relative to the body portion 1401. In this way, by being attached, the engagement member 1403 can be connected to the body portion 1401 (e.g., by circumferentially surrounding the body portion 1401) while not being limited to a single fixed position relative to the body portion 1401. Rather, in aspects, the engagement member 1403 can rotate relative to the body portion 1401, for example, about the hub axis 1003 along which the hub 56 extends. The body portion 1401 and the engagement member 1403 can circumferentially surround the hub axis 1003 and the central passage 801 while extending coaxially with one another. The engagement member 1403 can comprise an inner radial surface 1405 that surrounds a recess 1407 within which the body portion 1401 is received.

FIG. 15 is a sectional illustration of the hub 56 along lines 15-15 of FIG. 14, in which the engagement member 1403 is spaced apart from the body portion 1401 for the purposes of illustration and to more clearly show portions of the body portion 1401. The body portion 1401 can define and circumferentially surround the central passage 801, which may extend through the body portion 1401 along the hub axis 1003. In aspects, the body portion 1401 can define at least one wire opening 1501 extending radially through the body portion 1401 between the central passage 801 and an outer radial side 1503 of the body portion 1401. The at least one wire opening 1501 can extend through a wall of the body portion 1401 to define a passageway, path, etc., between the central passage 801 and an exterior of the body portion 1401 at the outer radial side 1503. In aspects, the at least one wire opening 1501 may extend along a radial axis that is substantially perpendicular to the hub axis 1003, however, any number of angles or orientations for the one wire opening 1501 can be provided. While FIG. 15 illustrates the at least one wire opening 1501 comprising two wire openings (e.g., a first wire opening 1507 and a second wire opening 1509) that are spaced circumferentially apart about 180 degrees about the hub axis 1003, more than two wire openings can be provided. The at least one wire opening 1501 can define a path through which the plurality of wires 825 can extend from the central passage 801 to an exterior of the body portion 1401. For example, a first wire 1511 of the plurality of wires 825 can extend through the first wire opening 1507, and a second wire 1513 of the plurality of wires 825 can extend through the second wire opening 1509. While FIG. 15 illustrates one wire passing through one wire opening, the wire openings may be sized to accommodate any number (e.g., one or more) of wires of the plurality of wires 825. In this way, one or more wires can pass through one wire opening.

The engagement member 1403 is substantially hollow, with the inner radial surface 1405 circumferentially surrounding the recess 1407. In aspects, the body portion 1401 can comprise a cross-sectional size (e.g., diameter) that is less than a cross-sectional size (e.g., diameter) of the recess 1407. In this way, the engagement member 1403 can be moved in a movement direction 1517 toward the body portion 1401, such that the body portion 1401 can pass through, and be received within, the recess 1407. In addition, or in the alternative, the body portion 1401 can be moved in a direction opposite the movement direction 1517 to cause the body portion 1401 to be through, and be received within, the recess 1407.

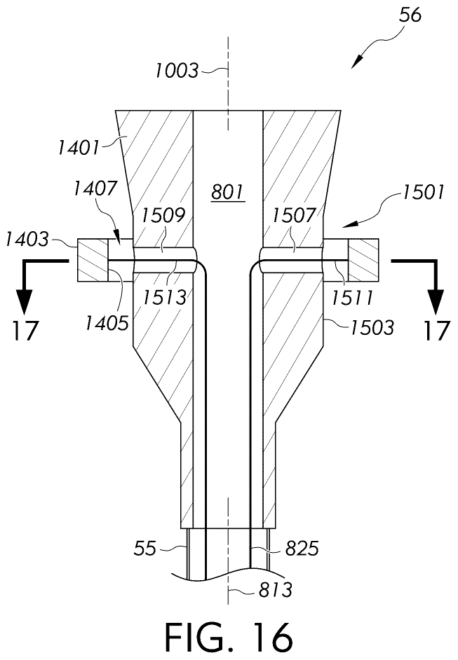

FIG. 16 is a sectional illustration similar to FIG. 15, but for the engagement member 1403 receiving the body portion 1401, and the plurality of wires 825 attached to the engagement member 1403. For example, the engagement member 1403 can be positioned at an axial location along the hub axis 1003 that substantially matches a position of the wire openings 1501. The diameter of the recess 1407 may be large enough such that the engagement member 1403 can rotate relative to the body portion 1401. In aspects, the first wire 1511 can extend from the central passage 801, through the first wire opening 1507, and into the recess 1407, whereupon the first wire 1511 is attached to the engagement member 1403. In aspects, the first wire 1511 may be attached at an end (e.g., second end 829) to a surface of the engagement member 1403, such as, for example, the inner radial surface 1405. Likewise, in aspects, the second wire 1513 can extend from the central passage 801, through the second wire opening 1509, and into the recess 1407, whereupon the second wire 1513 is attached to the engagement member 1403. In aspects, the second wire 1513 may be attached at an end (e.g., second end 829) to a surface of the engagement member 1403, such as, for example, the inner radial surface 1405. Accordingly, the plurality of wires 825 can extend through the at least one wire opening 1501 and may be attached to the engagement member 1403. The plurality of wires 825 can be attached in any number of ways to the engagement member 1403, for example, by adhesives, knots or other fastening mechanisms, etc.

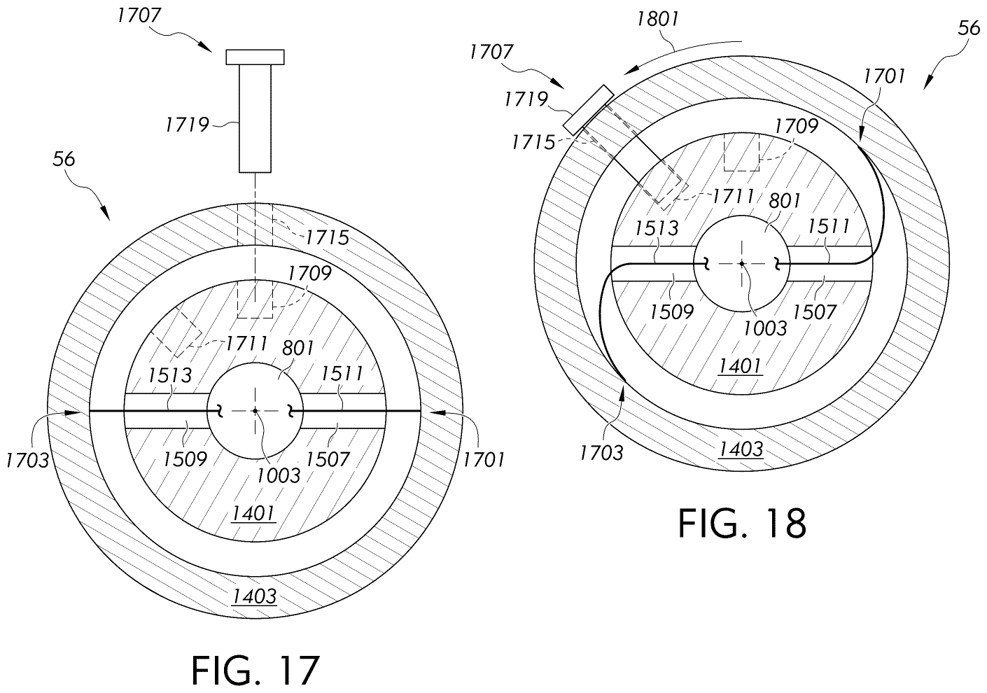

FIG. 17 illustrates a top-down sectional illustration of the hub 56 along lines 17-17 of FIG. 16, in which the wires 1511, 1513 extend from the central passage 801, through the wire openings 1507, 1509, and to the engagement member 1403. For example, FIG. 17 illustrates a position of the engagement member 1403 when the engagement member 1403 and the sheath 55 are in an extended position, which corresponds to the extended position illustrated in FIG. 8. In aspects, the first wire 1511 can be attached to the engagement member 1403 at a first attachment location 1701 of the engagement member 1403. The second wire 1513 can be attached to the engagement member 1403 at a second attachment location 1703 of the engagement member 1403. While FIG. 17 illustrates the engagement member 1403 as comprising two attachment locations 1701, 1703 that are spaced circumferentially apart about 180 degrees, any number of attachment locations (e.g., one or more) can be provided at varying locations along the engagement member 1403. Further, while FIG. 17 illustrates one wire attached to one attachment location, such a design is not intended to be limiting, as multiple wires can be attached to one attachment location.

Referring to FIG. 18, methods can comprise moving the plurality of wires 825 by rotating (e.g., illustrated with arrowhead 1801) the engagement member 1403 relative to the body portion 1401 such that the plurality of wires 825 and the distal sheath end 811 of the sheath 55 move axially in the proximal direction 835. The engagement member 1403 can be rotated 1801 at least until the engagement member 1403 and the sheath 55 are in a retracted position, which corresponds to the retracted position illustrated in FIG. 9. As illustrated in FIG. 18, rotation of the engagement member 1403 can cause the first attachment location 1701 and the second attachment location 1703 to move and rotate relative to the body portion 1401 about the hub axis 1003. In this way, the distance between the first attachment location 1701 and the first wire opening 1507 may increase as compared to the pre-rotation position illustrated in FIG. 17. Likewise, the distance between the second attachment location 1703 and the second wire opening 1509 may increase as compared to the pre-rotation position illustrated in FIG. 17. Due to the rotation of the first attachment location 1701 relative to the body portion 1401, the length of the portion of the first wire 1511 extending between the first wire opening 1507 and the first attachment location 1701 can increase. Similarly, due to the rotation of the second attachment location 1703 relative to the body portion 1401, the length of the portion of the second wire 1513 extending between the second wire opening 1509 and the second attachment location 1703 can also increase. As a result, the rotation of the engagement member 1403 can cause the plurality of wires 825 to move in the proximal direction 835 through the central passage 801, which can cause the distal sheath end 811 of the sheath 55 to move in the proximal direction 835. As such, the sheath 55 can move from the extended position, comprising the first cross-sectional dimension 817 and the first length 821 (e.g., illustrated in FIG. 8, which corresponds to the position illustrated in FIG. 17), to the retracted position, comprising the second cross-sectional dimension 901 and the second length 903 (e.g., illustrated in FIG. 9 which corresponds to the position illustrated in FIG. 18).

In aspects, and as illustrated in FIGS. 17-18, the hub 56 can comprise a locking mechanism 1707 that can selectively lock the engagement member 1403 relative to the body portion 1401 and prevent inadvertent or unintended rotation of the engagement member 1403 relative to the body portion 1401. For example, referring to FIG. 17, the locking mechanism 1707 can comprise one or more openings in the body portion 1401, for example, a first locking opening 1709, a second locking opening 1711, etc. The locking openings 1709, 1711 may be formed at an outer radial side of the body portion 1401. FIG. 17 illustrates two locking openings 1709, 1711 for the purposes of illustration, however, in aspects, additional locking openings (e.g., one or more) may be provided at the outer radial side of the body portion 1401 at several different locations. The locking mechanism 1707 can further comprise an engagement opening 1715 that extends radially through the engagement member 1403.

In aspects, the locking mechanism 1707 can comprise a locking structure 1719 that is sized and shaped to be received within the locking openings 1709, 1711 and the engagement opening 1715. For example, the locking structure 1719 can comprise a screw, a bolt, a detent pin, etc. The locking structure 1719 can pass through the engagement opening 1715 and may be received within one of the first locking opening 1709 or the second locking opening 1711. In aspects, to facilitate attachment of the locking structure 1719, one or more of the openings 1709, 1711, 1715 may be threaded and/or the locking structure 1719 may be threaded. In this way, the locking structure 1719 can be removably received within the one or more of the openings 1709, 1711, 1715 without being inadvertently removed. As illustrated in FIG. 18, in aspects, after rotation of the engagement member 1403, the locking structure 1719 can pass through the engagement opening 1715 and may be received within a locking opening, for example, the second locking opening 1711. As such, the locking mechanism 1707 can limit unintended rotation of the engagement member 1403 relative to the body portion 1401. The locking mechanism 1707 is not limited to the structure illustrated in FIGS. 17-18. Rather, in the alternative, the locking mechanism 1707 can comprise other structures or configurations that can selectively prevent rotation of the engagement member 1403 relative to the body portion 1401. For example, the locking mechanism 1707 can comprise a spring-loaded pin or locking structure 1719 housed in the body portion 1401 and projecting radially outwardly toward the engagement member 1403, wherein the spring-loaded pin can be received within the engagement opening 1715. It will be appreciated that while the locking mechanism 1707 is illustrated with respect to the embodiments of FIGS. 14-18, none, some, or all, of the other embodiments may comprise a locking mechanism that may be similar or identical to the locking mechanism 1707, or different than the locking mechanism 1707. In general, when the other embodiments comprise a locking mechanism, the locking mechanism can lock the engagement member relative to the body portion such that the sheath 55 can be held or locked in the extended position or retracted position (e.g., illustrated in FIGS. 8-9).

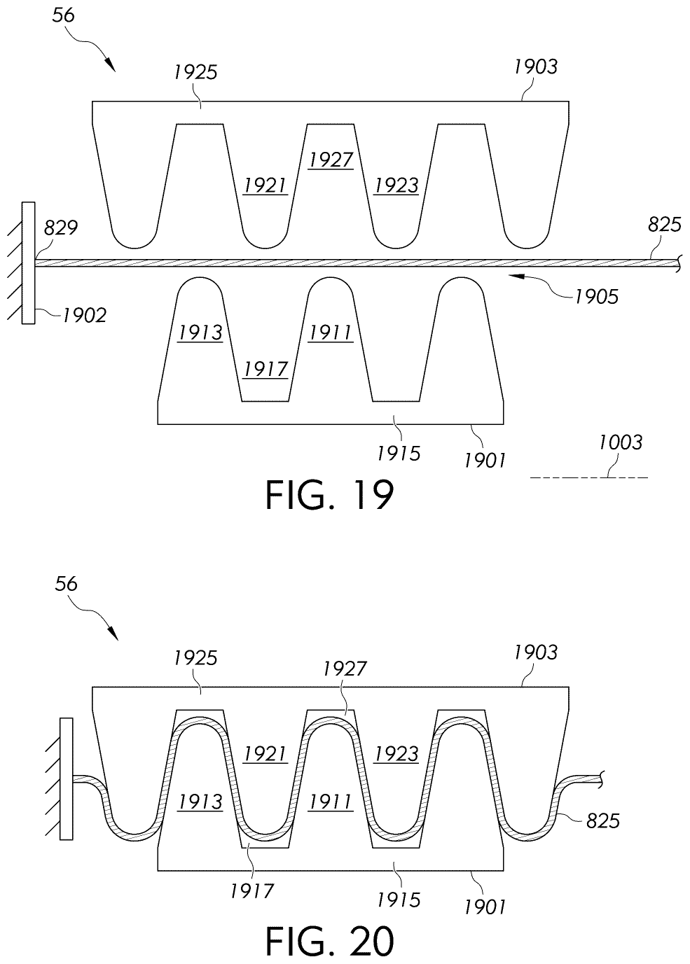

FIGS. 19-20 illustrate another embodiment of the hub 56 comprising a body portion 1901 and an engagement member 1903. For purposes of illustration, the sheath 55 is not illustrated in FIG. 19 so as to not obscure view of the plurality of wires 825. However, in operation, the sheath 55 is attached to the hub 56 as illustrated in FIGS. 8-9. As illustrated in FIG. 19, the body portion 1901 and the engagement member 1903 can be in a spaced-apart orientation, with the plurality of wires 825 extending through a gap 1905 defined between the body portion 1901 and the engagement member 1903. FIG. 19 illustrates a position of the engagement member 1903 relative to the body portion 1901 when the engagement member 1903 and the sheath 55 are in an extended position, which corresponds to the extended position illustrated in FIG. 8. As illustrated in FIG. 19, when the body portion 1901 and the engagement member 1903 are in the spaced-apart orientation, the plurality of wires 825 can extend along a linear travel path through the gap 1905. The second end 829 of the plurality of wires 825 can be attached to a fixed structure 1902 (e.g., a portion of the body portion 1901, etc.) that maintains a position of the plurality of wires 825 within the gap 1905.

In aspects, the body portion 1901 can comprise one or more body projections, such as, for example, a first body projection 1911, a second body projection 1913, etc. The body projections 1911, 1913 can project outwardly from a wall 1915 of the body portion 1901 and may project toward the engagement member 1903. The body projections 1911, 1913 can be spaced apart with a first body channel 1917 defined between the first body projection 1911 and the second body projection 1913. In aspects, the body projections 1911, 1913 can comprise a peak-like shape that each converge toward central points in a direction away from the wall 1915. That is, adjacent to the wall 1915, the body projections 1911, 1913 comprise a maximum cross-sectional dimension, and may decrease in cross-sectional dimension in a direction away from the wall 1915. While the body portion 1901 is illustrated as comprising three body projections and two body channels in FIG. 19, any number of body projections and body channels may be provided. The body projections are spaced apart with a body channel defined between two neighboring body projections, such that the body portion 1901 comprises alternating body projections and body channels along the wall 1915.

In aspects, the engagement member 1903 can comprise one or more engagement projections, such as, for example, a first engagement projection 1921, a second engagement projection 1923, etc. The engagement projections 1921, 1923 can project outwardly from a wall 1925 of the engagement member 1903 and may project toward the body portion 1901. The engagement projections 1921, 1923 can be spaced apart with a first engagement channel 1927 defined between the first engagement projection 1921 and the second engagement projection 1923. In aspects, the engagement projections 1921, 1923 can comprise a peak-like shape that converges toward a central point in a direction away from the wall 1925. That is, adjacent to the wall 1925, the engagement projections 1921, 1923 comprise a maximum cross-sectional dimension, and may decrease in cross-sectional dimension in a direction away from the wall 1925. While the engagement member 1903 is illustrated as comprising four engagement projections and three engagement channels in FIG. 19, any number of engagement projections and engagement channels may be provided. The engagement projections may be spaced apart with an engagement channel defined between two neighboring engagement projections, such that the engagement member 1903 comprises alternating engagement projections and engagement channels along the wall 1925. In aspects, the size and shape of the engagement projections 1921, 1923 may substantially match a size and shape of the body projections 1911, 1913, and the size and shape of the body channels 1917 can substantially match a size and shape of the engagement channels 1927. In aspects, other shapes of the body projections 1911, 1913 and the engagement projections 1921, 1923 can be provided, for example, with the body projections 1911, 1913 comprising steps, teeth, or ridges that match and engage with corresponding steps, teeth, or ridges of the and the engagement projections 1921, 1923. In this way, additional control over the movement of the plurality of wires 825 can be provided.

In aspects, the body projections 1911, 1913 may be offset from the engagement projections 1921, 1923 along the hub axis 1003. In this way, a body projection can be received within an engagement channel. Likewise, an engagement projection can be received within a body channel. For example, as illustrated in FIG. 20, methods can comprise moving the plurality of wires 825 by moving the engagement member 1903 and the body portion 1901 together to compress at least one wire 825 such that the at least one wire 825 extends along a non-linear travel path. Moving the engagement member 1903 and the body portion 1901 can comprise moving the engagement member 1903 relative to the body portion 1901 (e.g., by moving the engagement member 1903 toward the body portion 1901), moving the body portion 1901 relative to the engagement member 1903 (e.g., by moving the body portion 1901 toward the engagement member 1903), and/or moving both the engagement member 1903 and the body portion 1901 (e.g., by moving the engagement member 1903 toward the body portion 1901 concurrently with moving the body portion 1901 toward the engagement member 1903). In this way, the body projections 1911, 1913 can be received within engagement channels 1927, and engagement projections 1921, 1923 can be received within body channels 1917. For example, the first body projection 1911 can be received within the first engagement channel 1927, which can likewise cause one or more wires of the plurality of wires 825 to move from the linear path (e.g., illustrated in FIG. 18) to moving along a non-linear path and extending within the first engagement channel 1927. Likewise, the first engagement projection 1921 can be received within the first body channel 1917, which can likewise cause one or more wires of the plurality of wires 825 to move from the linear path (e.g., illustrated in FIG. 18) to moving along a non-linear path and extending within the first body channel 1917. As illustrated herein, in aspects, the projections 1911, 1913, 1921, 1923 can comprise a rounded or blunt shape or tip to reduce the likelihood of damage to the wires 825. That is, the end of the projections 1911, 1913, 1921, 1923 that contacts the wires 825 may have the rounded or blunt shape.

Accordingly, the engagement member 1903 and the body portion 1901 can move relative to one another between a first position (e.g., illustrated in FIG. 18) and a second position (e.g., illustrated in FIG. 19). In the first position, the engagement member 1903 is spaced apart from the body portion 1901 such that the first body projection 1911 is not received within the first engagement channel 1927 (e.g., or any engagement channel), and at least one wire of the plurality of wires 825 extends between the engagement member 1903 and the body portion 1901 (e.g., along a linear travel path and outside of the body channels or engagement channels). In the second position, the first body projection 1911 is received within the first engagement channel 1927, and at least one wire of the plurality of wires 825 extends within the engagement channels 1927 and the body channels 1917 along the non-linear travel path. As a result, due to the plurality of wires 825 being contacted and moved by the body portion 1901 and the engagement member 1903, the plurality of wires 825 can move in the proximal direction 835, which can cause the distal sheath end 811 of the sheath 55 to move in the proximal direction 835. As such, the sheath 55 can move from the extended position, comprising the first cross-sectional dimension 817 and the first length 821 (e.g., illustrated in FIG. 8, which corresponds to the position illustrated in FIG. 19), to the retracted position, comprising the second cross-sectional dimension 901 and the second length 903 (e.g., illustrated in FIG. 9 which corresponds to the position illustrated in FIG. 20).

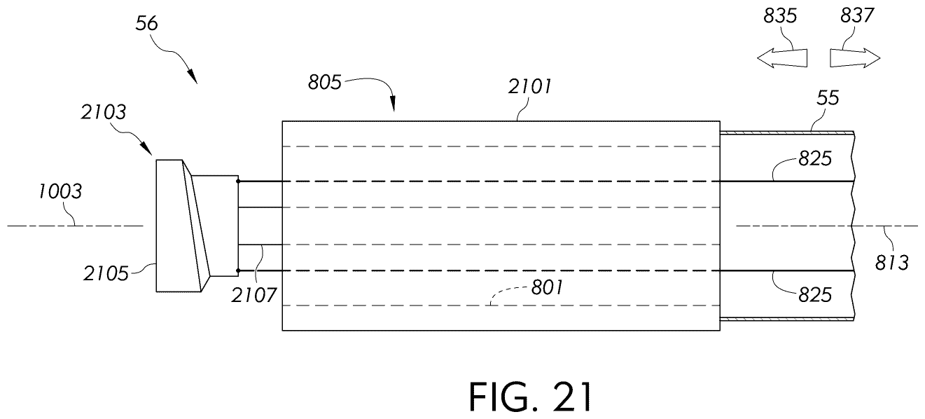

FIG. 21 illustrates another embodiment of the hub 56, wherein the hub 56 comprises a body portion 2101 and an engagement member 2103. In aspects, the body portion 2101 can at least partially receive a portion of the engagement member 2103 within the central passage 801. For example, the engagement member 2103 can comprise a handle 2105 and a slider 2107. The handle 2105 and the slider 2107 can be attached to one another and may extend along the axis 1003. In aspects, the slider 2107 can project outwardly from the handle 2105 and may be received within the central passage 801, while the handle 2105 may be located at an exterior of the central passage 801. In aspects, the plurality of wires 825 may be attached to one, or both, of the handle 2105 or the slider 2107. In operation, a user can manipulate the handle 2105 and move the handle 2105 relative to the body portion 2101, wherein movement of the handle 2105 can likewise cause movement of the slider 2107. For example, the handle 2105 can be moved in the proximal direction 835. Due to the plurality of wires 825 being attached to the engagement member 2103 (e.g., to one or more of the handle 2105 or the slider 2107), the plurality of wires 825 can move in the proximal direction 835, which can cause the distal sheath end 811 of the sheath 55 to move in the proximal direction 835. As such, the sheath 55 can move from the extended position, comprising the first cross-sectional dimension 817 and the first length 821 (e.g., illustrated in FIG. 8), to the retracted position, comprising the second cross-sectional dimension 901 and the second length 903 (e.g., illustrated in FIG. 9).

Aspect 1. An introducer apparatus is provided for delivering a heart valve prosthesis to a treatment site. The introducer apparatus comprises a hub comprising a central passage and an engagement member. The introducer apparatus comprises a sheath attached to the hub and comprising a first cross-sectional dimension along a plane that is perpendicular to a central axis of the sheath. The introducer apparatus comprises a plurality of wires extending between a first end and an opposing second end. The first end is attached to a distal sheath end of the sheath and the second end is attached to the engagement member. The engagement member is configured to move the plurality of wires and the distal sheath end between an extended position, in which the sheath comprises the first cross-sectional dimension, and a retracted position, in which the sheath comprises a second cross-sectional dimension that is greater than the first cross-sectional dimension.

Aspect 2. The introducer apparatus of aspect 1, wherein the engagement member is attached to a body portion of the hub, the engagement member configured to move relative to the body portion.

Aspect 3. The introducer apparatus of any of aspects 1-2, wherein the engagement member comprises a first engagement portion that is rotatable relative to the body portion, the first engagement portion circumferentially surrounding the central passage; and a second engagement portion received within the first engagement portion, the second engagement portion configured to move axially relative to the first engagement portion.

Aspect 4. The introducer apparatus of any of aspects 1-3, wherein the second end of the plurality of wires is attached to the second engagement portion such that the plurality of wires extend along the sheath.

Aspect 5. The introducer apparatus of any of aspects 1-4, wherein the engagement member circumferentially surrounds the body portion.

Aspect 6. The introducer apparatus of any of aspects 1-5, wherein the body portion defines at least one wire opening extending radially through the body portion between the central passage and an outer radial side of the body portion, the plurality of wires extending through the at least one wire opening such that the second end is attached to the engagement member.

Aspect 7. The introducer apparatus of any of aspects 1-6, wherein the engagement member is configured to rotate relative to the body portion, and wherein a locking mechanism contacts the engagement member and the body portion to selectively prevent rotation of the engagement member relative to the body portion.

Aspect 8. The introducer apparatus of any of aspects 1-7, wherein the engagement member comprises a first engagement projection and a second engagement projection that are spaced apart to define a first engagement channel between the first engagement projection and the second engagement projection.

Aspect 9.The introducer apparatus of any of aspects 1-8, wherein the body portion comprises a first body projection that is configured to be received within the first engagement channel such that the engagement member and the body portion are configured to move relative to one another between: a first position, in which the engagement member is spaced apart from the body portion such that the first body projection is not received within the first engagement channel, and at least one wire of the plurality of wires extends between the engagement member and the body portion; and a second position, in which the first body projection is received within the first engagement channel, and at least one wire of the plurality of wires extends within the first engagement channel.

Aspect 10. An introducer apparatus is provided for delivering a heart valve prosthesis to a treatment site. The introducer apparatus comprises a hub comprising a body portion surrounding a central passage, and an engagement member attached to the body portion and configured to move relative to the body portion. The introducer apparatus comprises a sheath attached to the hub. The sheath comprises a lumen in communication with the central passage. The sheath comprises a first cross-sectional dimension along a plane that is perpendicular to a central axis of the sheath. A plurality of wires extend between a first end and an opposing second end. The first end is attached to a distal sheath end of the sheath and the second end is attached to the engagement member. The engagement member is configured to move the plurality of wires and the distal sheath end from an extended position, in which the sheath comprises the first cross-sectional dimension and a first length, to a retracted position, in which the sheath comprises a second cross-sectional dimension, which is greater than the first cross-sectional dimension, and a second length, which is less than the first length.

Aspect 11. The introducer apparatus of aspect 10, wherein the engagement member comprises: a first engagement portion that is rotatable relative to the body portion, the first engagement portion circumferentially surrounding the central passage; and a second engagement portion received within the first engagement portion, the second engagement portion configured to move axially relative to the first engagement portion, the second end of the plurality of wires attached to the second engagement portion such that the plurality of wires extend along the sheath.

Aspect 12. The introducer apparatus of any of aspects 10-11, wherein the engagement member circumferentially surrounds the body portion and is configured to rotate relative to the body portion, and wherein the body portion defines at least one wire opening extending radially through the body portion between the central passage and an outer radial side of the body portion, the plurality of wires extending through the at least one wire opening such that the second end is attached to the engagement member.

Aspect 13. The introducer apparatus of any of aspects 10-12, wherein the engagement member comprises a first engagement projection and a second engagement projection that are spaced apart to define a first engagement channel between the first engagement projection and the second engagement projection, and wherein the body portion comprises a first body projection that is configured to be received within the first engagement channel, at least one wire of the plurality of wires extending between the engagement member and the body portion.

Aspect 14. Methods of delivering a heart valve prosthesis to a treatment site comprising providing a sheath attached to a hub, the sheath comprising a first cross-sectional dimension along a plane that is perpendicular to a central axis of the sheath. Methods comprise moving a plurality of wires that are attached to a distal sheath end of the sheath in a proximal direction toward the hub. Methods comprise increasing a cross-sectional dimension of the sheath.

Aspect 15. The method of aspect 14, wherein moving the plurality of wires comprises rotating a first engagement portion relative to a body portion of the hub such that a second engagement portion, which is received within the first engagement portion, moves axially relative to the first engagement portion.

Aspect 16. The method of any one of aspects 14-15, wherein the plurality of wires are attached to the second engagement portion such that the axial movement of the second engagement portion causes the plurality of wires and the distal sheath end of the sheath to move in the proximal direction.

Aspect 17. The method of any one of aspects 14-16, wherein the plurality of wires extend through at least one wire opening of a body portion of the hub and are attached to an engagement member of the hub.

Aspect 18. The method of any one of aspects 14-17, wherein moving the plurality of wires comprises rotating the engagement member relative to the body portion such that the plurality of wires and the distal sheath end of the sheath move axially in the proximal direction.

Aspect 19. The method of any one of aspects 14-18, wherein at least one wire of the plurality of wires is positioned between an engagement member of the hub and a body portion of the hub, the at least one wire extending along a linear travel path.

Aspect 20. The method of any one of aspects 14-19, wherein moving the plurality of wires comprises moving the engagement member and the body portion together to compress the at least one wire such that the at least one wire extends along a non-linear travel path.

It should be understood that while various aspects have been described in detail relative to certain illustrative and specific examples thereof, the present disclosure should not be considered limited to such, as numerous modifications and combinations of the disclosed features are possible without departing from the scope of the following claims.

What is claimed is:

1. An introducer apparatus for delivering a heart valve prosthesis to a treatment site, the introducer apparatus comprising:
a hub comprising a central passage and an engagement member;
a sheath attached to the hub and comprising a first cross-sectional dimension along a plane that is perpendicular to a central axis of the sheath; and
a plurality of wires extending between a first end and an opposing second end, the first end attached to a distal sheath end of the sheath and the second end attached to the engagement member, the engagement member configured to move the plurality of wires and the distal sheath end between:
an extended position, in which the sheath comprises the first cross-sectional dimension; and
a retracted position, in which the sheath comprises a second cross-sectional dimension that is greater than the first cross-sectional dimension.

2. The introducer apparatus of claim 1, wherein the engagement member is attached to a body portion of the hub, the engagement member configured to move relative to the body portion.

3. The introducer apparatus of claim 2, wherein the engagement member comprises:
a first engagement portion that is rotatable relative to the body portion, the first engagement portion circumferentially surrounding the central passage; and
a second engagement portion received within the first engagement portion, the second engagement portion configured to move axially relative to the first engagement portion.

4. The introducer apparatus of claim 3, wherein the second end of the plurality of wires are attached to the second engagement portion such that the plurality of wires extend along the sheath.

5. The introducer apparatus of claim 2, wherein the engagement member circumferentially surrounds the body portion.

6. The introducer apparatus of claim 5, wherein the body portion defines at least one wire opening extending radially through the body portion between the central passage and an outer radial side of the body portion, the plurality of wires extending through the at least one wire opening such that the second end is attached to the engagement member.

7. The introducer apparatus of claim 6, wherein the engagement member is configured to rotate relative to the body portion, and wherein a locking mechanism contacts the engagement member and the body portion to selectively prevent rotation of the engagement member relative to the body portion.

8. The introducer apparatus of claim 2, wherein the engagement member comprises a first engagement projection and a second engagement projection that are spaced apart to define a first engagement channel between the first engagement projection and the second engagement projection.

9. The introducer apparatus of claim 8, wherein the body portion comprises a first body projection that is configured to be received within the first engagement channel such that the engagement member and the body portion are configured to move relative to one another between:
a first position, in which the engagement member is spaced apart from the body portion such that the first body projection is not received within the first engagement channel, and at least one wire of the plurality of wires extends between the engagement member and the body portion; and
a second position, in which the first body projection is received within the first engagement channel, and at least one wire of the plurality of wires extends within the first engagement channel.

10. An introducer apparatus for delivering a heart valve prosthesis to a treatment site, the introducer apparatus comprising:
a hub comprising:
a body portion surrounding a central passage; and
an engagement member attached to the body portion and configured to move relative to the body portion;
a sheath attached to the hub, the sheath comprising a lumen in communication with the central passage, the sheath comprising a first cross-sectional dimension along a plane that is perpendicular to a central axis of the sheath; and
a plurality of wires extending between a first end and an opposing second end, the first end attached to a distal sheath end of the sheath and the second end attached to the engagement member, the engagement member configured to move the plurality of wires and the distal sheath end from an extended position, in which the sheath comprises the first cross-sectional dimension and a first length, to a retracted position, in which the sheath comprises a second cross-sectional dimension, which is greater than the first cross-sectional dimension, and a second length, which is less than the first length.

11. The introducer apparatus of claim 10, wherein the engagement member comprises:
a first engagement portion that is rotatable relative to the body portion, the first engagement portion circumferentially surrounding the central passage; and
a second engagement portion received within the first engagement portion, the second engagement portion configured to move axially relative to the first engagement portion, the second end of the plurality of wires attached to the second engagement portion such that the plurality of wires extend along the sheath.

12. The introducer apparatus of claim 10, wherein the engagement member circumferentially surrounds the body portion and is configured to rotate relative to the body portion, and wherein the body portion defines at least one wire opening extending radially through the body portion between the central passage and an outer radial side of the body portion, the plurality of wires extending through the at least one wire opening such that the second end is attached to the engagement member.

13. The introducer apparatus of claim 10, wherein the engagement member comprises a first engagement projection and a second engagement projection that are spaced apart to define a first engagement channel between the first engagement projection and the second engagement projection, and wherein the body portion comprises a first body projection that is configured to be received within the first engagement channel, at least one wire of the plurality of wires extending between the engagement member and the body portion.

14. A method of delivering a heart valve prosthesis to a treatment site comprising:
providing a sheath attached to a hub, the sheath comprising a first cross-sectional dimension along a plane that is perpendicular to a central axis of the sheath;
moving a plurality of wires that are attached to a distal sheath end of the sheath in a proximal direction toward the hub; and
increasing a cross-sectional dimension of the sheath.

15. The method of claim 14, wherein moving the plurality of wires comprises rotating a first engagement portion relative to a body portion of the hub such that a second engagement portion, which is received within the first engagement portion, moves axially relative to the first engagement portion.

16. The method of claim 15, wherein the plurality of wires are attached to the second engagement portion such that the axial movement of the second engagement portion causes the plurality of wires and the distal sheath end of the sheath to move in the proximal direction.

17. The method of claim 14, wherein the plurality of wires extend through at least one wire opening of a body portion of the hub and are attached to an engagement member of the hub.

18. The method of claim 17, wherein moving the plurality of wires comprises rotating the engagement member relative to the body portion such that the plurality of wires and the distal sheath end of the sheath move axially in the proximal direction.

19. The method of claim 14, wherein at least one wire of the plurality of wires is positioned between an engagement member of the hub and a body portion of the hub, the at least one wire extending along a linear travel path.

20. The method of claim 19, wherein moving the plurality of wires comprises moving the engagement member and the body portion together to compress the at least one wire such that the at least one wire extends along a non-linear travel path.
